(19) United States
(12) Patent Application Publication
(10) Pub. No.: US 2004/0039355 A1
(43) Pub. Date: Feb. 26, 2004

(54) Fluid dispensing devices and methods

(76) Inventors: Jose M. Gonzalez, Aquadilla, PR (US); Orlando E. Ruiz, Rincon, PR (US)

Correspondence Address: HEWLETT-PACKARD COMPANY, Intellectual Property Administration, P.O. Box 272400, Fort Collins, CO, 80527-2400, US

(21) Appl. No.: 10/227,736

(22) Filed: Aug. 26, 2002

(51) Int. Cl. A61M 35/00

(52) U.S. Cl. 604/298

(57) ABSTRACT

Fluid dispensing devices and methods according to specific embodiments of the invention include a device for ejecting or otherwise dispensing medication, for example eye medication, or other fluid or substance. A thermal ejector is optionally provided to aid in the dispensing. A control device, for example a microprocessor, controls the thermal ejector. Communications to and from a remote location are also optionally provided.

### BACKGROUND OF THE INVENTION

Eyedroppers are used to dispense one or more drops of eye medication. With the most common form of eyedropper, a user must tilt his or her head far back such that the eye is in a horizontal or near-horizontal orientation. The patient maneuvers the eyedropper into position over the eye and squeezes a bulb or other compressible member such that a drop emerges and free falls into the eye. Tilting the head back can be distracting and potentially dangerous in certain situations, for example while driving an automobile. Additionally, the gravity-induced free fall of the drop can be difficult to control, resulting in drops missing the eye and instead hitting the user's face or other surface. The user thus wastes the medication being dispensed. A child user may be unwilling or unable to use a typical eyedropper properly or at all. Additionally, if the user fails to accurately place a complete drop into the eye, or places too many drops into the eye, the intended benefits of the medication may be diminished or lost.

Drop-on-demand inkjet printers use printhead nozzles that each eject a single drop of ink only when activated. Thermal inkjet and piezoelectric inkjet are two common drop-on-demand inkjet technologies. Thermal inkjet printers use heat to generate vapor bubbles, ejecting small drops of ink through nozzles and placing them precisely on a surface to form text or images. Advantages of thermal inkjet printers include small drop sizes, high printhead operating frequency, excellent system reliability and highly controlled ink drop placement. Integrated electronics mean fewer electrical connections, faster operation and higher color resolution. Originally developed for desktop printers, thermal inkjet technology is designed to be inexpensive, quiet and easy to use.

### SUMMARY OF THE INVENTION

Fluid dispensing devices and methods according to specific embodiments of the invention include a device for ejecting or otherwise dispensing medication, for example eye medication, or other fluid or substance. A thermal ejector is optionally provided to aid in the dispensing. A control device, for example a microprocessor, controls the thermal ejector. Communications to and from a remote location are also optionally provided. Features according to other specific embodiments are described in the remainder of this patent application.

### BRIEF DESCRIPTION OF THE DRAWINGS

The accompanying drawings illustrate embodiments of the present invention and together with the description serve to explain certain principles of the invention. Other embodiments of the present invention will be readily appreciated with reference to the drawings and the description, in which like reference numerals designate like parts and in which:

FIG. 1 is a block diagram of an eye drop device according to an embodiment of the invention;

FIG. 2 is a perspective view of an eye drop device with first and second members in a separated state, according to an embodiment of the invention;

FIG. 3 is a side view of an eye drop device in use, according to an embodiment of the invention;

FIG. 4 a perspective, partially cut-away view of a thermal ejection nozzle according to an embodiment of the invention;

FIG. 5 is a side view of the FIG. 4 nozzle;

FIGS. 6-9 are perspective views of the FIG. 4 nozzle in different stages of drop formation and ejection, according to an embodiment of the invention;

FIGS. 10-11 are side views of a piezoelectric nozzle, according to an embodiment of the invention;

FIG. 12 is a schematic diagram showing communication features according to embodiments of the invention;

FIG. 13 is an additional schematic diagram showing communication features according to embodiments of the invention; and

FIG. 14 is a side view of an eye drop device according to an embodiment of the invention.

### DETAILED DESCRIPTION OF THE PREFERRED EMBODIMENTS

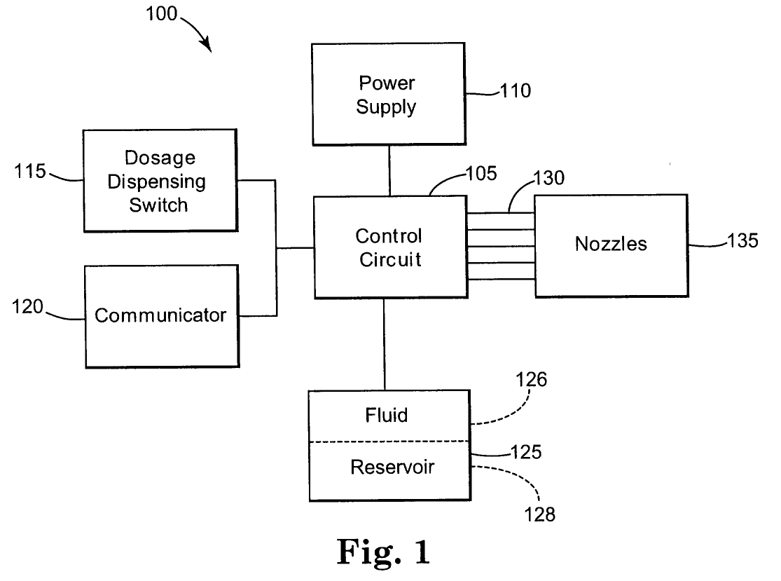

FIG. 1 is a block diagram of eyedropper device 100 according to an embodiment of the invention. Device 100 includes control circuit 105, which is or which includes a microprocessor, a microcontroller and/or a computing device that is operably coupled with the other elements of device 100 to be described herein. Control circuit 105 is programmed or otherwise adapted to operate a fluid ejection head in response to control inputs, and to perform other functions as will be described. Power supply 110, such as a battery pack, provides power to control circuit 105.

Dosage dispensing switch 115 is connected to control circuit 105 and is adapted for manual actuation by a user to initiate or control dispensing of fluid or other substance from device 100. In alternative embodiments, multiple different switches 115 or other controls are provided and connected to control circuit 105, to achieve different optional functions. An example of one such function is controlling which of several types of medication is to be dispensed. Actuation of dosage switch 115 causes a predetermined amount or dosage of medication or other substance to be dispensed, according to embodiments of the invention. According to additional embodiments, medication or other substance is dispensed for as long as switch 115 is depressed, such that the user controls dosage more directly.

Dosage dispensing switch 115 and control circuit 105 also are connected to communicator 120, which for example comprises a computer port, communications port, USB port or other device for communicating electrical, optical, hard-wired, wireless or other signals or information, in a manner to be described. Control circuit 105 also is operably coupled with fluid reservoir 125, which optionally includes two or more separate chambers 126, 128 for holding different types of fluid or other substance.

One or more communication lines, in the form of e.g. multi-line control bus 130, provide electronic communication to one or more nozzles 135, for example an array of nozzles. In one embodiment, control bus 130 has 16 lines, and device 100 operates in a multiplexed manner such that each of several hundred nozzles 135 is individually addressed. Certain aspects of control circuit 105 operate in generally the same manner as a controller for a thermal or piezoelectric inkjet printer, if desired, although simplification of those aspects is also contemplated. For example, control circuit 105 is optionally constructed without the capability to address individual nozzles, or without the capability to change the specific nozzles that are operating from moment to moment. Thus, control circuit 105 is optionally programmed with relatively straightforward software, within the knowledge of e.g. one skilled in the art of inkjet printers. Alternatively, control circuit 105 optionally includes more complex programming than that usually associated with inkjet printers.

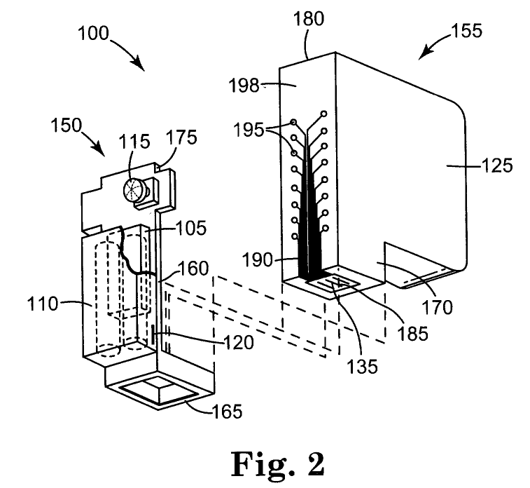

FIG. 2 shows first portion 150 and second portion 155 of apparatus 100. First portion 150 includes main panel 160, which supports control circuit 105, power supply 110, dosage dispensing switch 115, and communicator 120. In the FIG. 2 embodiment, communicator 120 is in the form of a USB port. First portion 150 can support other features, such as an LCD or other display for displaying dosage information, type of medication dispensed, time and date of previous dose or time elapsed since previous dose, alarm indications, reminders, instructions to contact a medical provider, and other indications that are potentially relevant to the user of device 100. According to specific embodiments of the invention, control circuit 105 is an integrated circuit chip in a carrier mounted to panel 160, and power supply 110 comprises two batteries contained in a compartment connected to panel 160. Other control circuit and power supply embodiments are also contemplated.

First portion 150 is permanently or readily removably attached to second portion 155. Nose 165 of first portion 150 surrounds and holds nose 170 of second portion 155, and angled or rectangular edge or latch 175 catches a corresponding edge 180 of second portion 155 to secure portions 150, 155 together, according to the illustrated embodiment.

Second portion 155 includes fluid reservoir 125, fluidly connected to nozzles 135 of fluid ejection head 185. When first portion 150 and second portion 155 are connected, nose 165 of first portion 150 surrounds, contains or supports fluid ejection head 185, according to the illustrated embodiment. Electrical connector 190, comprising multiple conductive traces, for example, connects nozzles 135 of head 185 with array 195 of conductive pads. Array 195 wraps along one edge face 198 of second portion 155. Control circuit 105 controls fluid ejection head 185 through electrical connection with array 195 and connector 190. Device 100 also optionally includes conductors that connect contacts, switches, batteries and other electrical components to control circuit 105. These conductors are optionally in the form of wires, one or more flexible circuits, one or more printed circuit boards, or other suitable alternatives, including conductors formed as part of the body of device 100. Second portion 155 is generally in the form of a cartridge, for example an ink jet pen, according to embodiments of the invention.

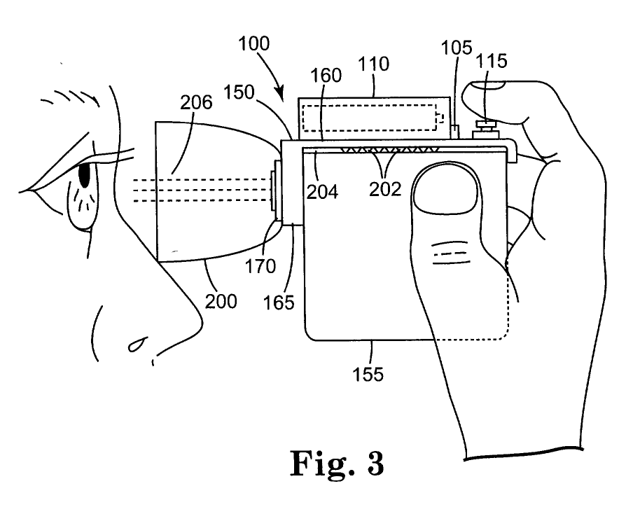

FIG. 3 shows first portion 150 and second portion 155 connected together and held in the hand of a user. FIG. 3 also illustrates eye cup 200 for maintaining a desired, generally constant distance between fluid ejection head 185 and the eye of a patient. If that distance is too great, it is possible for drops to partially or entirely miss the eye and thus not be administered properly. If that distance is too small, eye injury or irritation is possible due to the force with which drops are ejected from head 185, and/or the area over which drops contact the eye is possibly too small. Therefore, eye cup 200 is optionally configured to generally prevent a user from moving fluid ejection head 185 too close to the eye, i.e. to generally require or maintain a minimum separation or distance between fluid ejection head 185 and the eye. Accordingly, eye cup 200 extends between ejection head 185 and the eye. Eye cup 200 is connected to first portion 150, for example by snap-fitting, gluing, welding, or otherwise securing eye cup 200 to nose 165. Alternatively, or additionally, eye cup 200 is attached to nose 170 of second portion 155, which extends through nose 165 of first portion 150 as shown.

FIG. 3 also shows conductive contacts 202 located at rear surface 204 of panel 160. Contacts 202 are positioned to register with array 195 of conductive pads of second portion 155 and provide electrical communication e.g. between control circuit 105 and fluid ejection head 185.

As also shown in FIG. 3, a user holds device 100 in one hand and actuates switch 115, for example with one finger, to emit one or more droplets 206 of eye medication, fluid or other substance toward his or her eye. Droplets 206 are fired with enough velocity that they maintain a trajectory that is not significantly affected by gravity, according to embodiments of the invention. Device 100 is entirely self-contained, according to embodiments of the invention, with the medication or other substance to be dispensed, battery power, and processing power all on-board. Device 100 reduces or eliminates the need for power or fluid supply conduits to connect to a remote base. In alternative embodiments, device 100 is supplemented with either power or fluid provided from a remote supply.

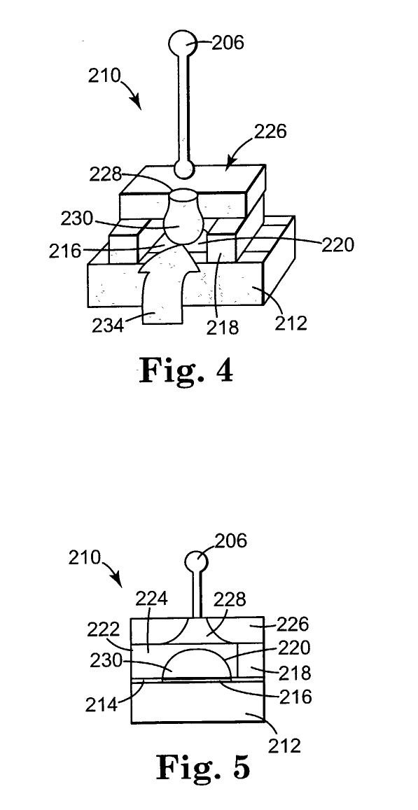

Specific embodiments of nozzles 135 of fluid ejection head 185, and their actuation, now are described with respect to e.g. FIGS. 4-11. FIGS. 4-5 illustrate a single nozzle 210 in the array of nozzles 135. Nozzle 210 includes silicon substrate 212 that supports thin-film conductor 214 and thin-film resistor 216. An opening in photoimageable polymer barrier 218 defines firing chamber 220, which is fluidly coupled with channel 222 for holding eye medication 224 or other fluid or substance (hereinafter “fluid,” to simplify the disclosure) to be dispensed. Orifice plate 226 defines channel orifice 228. Resistor 216 is located in the center of the floor of firing chamber 220, and upon application of electricity rapidly heats a thin layer of fluid 224. Resistor 216 thus is one example of a heating element according to an embodiment of the invention, and acts as an actuator of a thermal pump or thermal ejector according to an embodiment of the invention. The thermal pump or ejector is considered to include one or more of resistor 216, firing chamber 220, and orifice 228, and/or other features of nozzle 210, or nozzle 210 itself, according to embodiments of the invention, as well as additional features if desired. A tiny fraction of fluid 224 is vaporized to form expanding bubble 230 that ejects droplet 206 of fluid, for example toward the eye of a human user or patient. Refill fluid 234 or is drawn into firing chamber 220 automatically for subsequent droplet formation and ejection. Multiple nozzles 210 generally are disposed for ejecting fluid droplets through multiple orifices 228 in a single orifice plate 226, according to embodiments of the invention. In the case where first and second portions 150, 155 are connected together to form a common housing, a thermal pump or ejector according to an embodiment of the invention and the microprocessor or other control-circuit 105 feature are together located in the common housing. It is also contemplated that the thermal pump, control circuit 105 and/or other desired features are optionally provided within first portion 150 alone or second portion 155 alone, and thus located within a common housing. Other structural layouts and designs according to embodiments of the invention will be apparent to those of ordinary skill upon reading this disclosure.

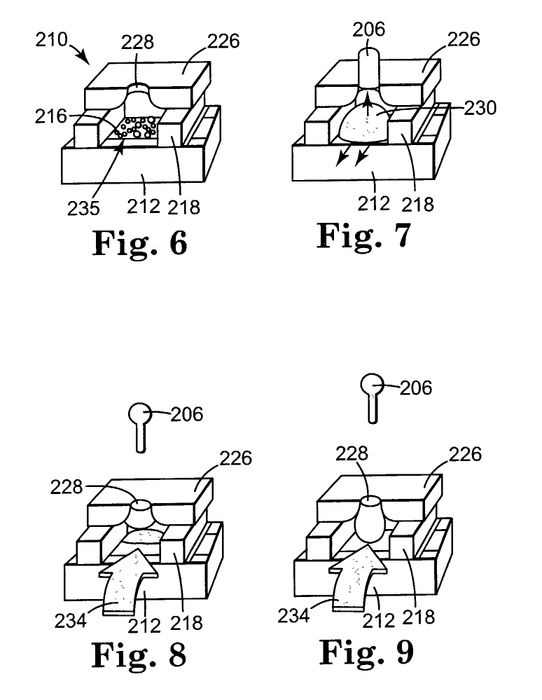

According to one specific example shown in FIGS. 6-9, resistor 216 heats fluid at more than one hundred degrees Centigrade per microsecond, causing film boiling shown generally at 235 in FIG. 6 in less than about 3 microseconds. Bubble 230 expands, forming droplet 206 as shown in FIG. 7, at about 3-10 microseconds from start. Bubble collapse and drop break-off occur at about 10-20 microseconds from start, as shown in FIG. 8, ejecting droplet 206 and drawing in fresh refill fluid 234. A fluid meniscus in orifice 228 settles and refill completes, as shown in FIG. 9, in less than about 80 microseconds from start. Refill and firing thus occur as fast as about 12,500 kHz or faster. Nozzle 210 heats a thin film of fluid about 0.1 micrometers thick to about 340 degrees Celsius, according to one embodiment. Other temperatures, thicknesses, volumes and frequencies are also contemplated. In some cases, the relatively high temperatures created by resistor or other heating element 216 optionally are used to at least partially sterilize medication before it is ejected. The expanding vapor bubble 230 forms to expel the fluid. No moving parts are used except the fluid itself.

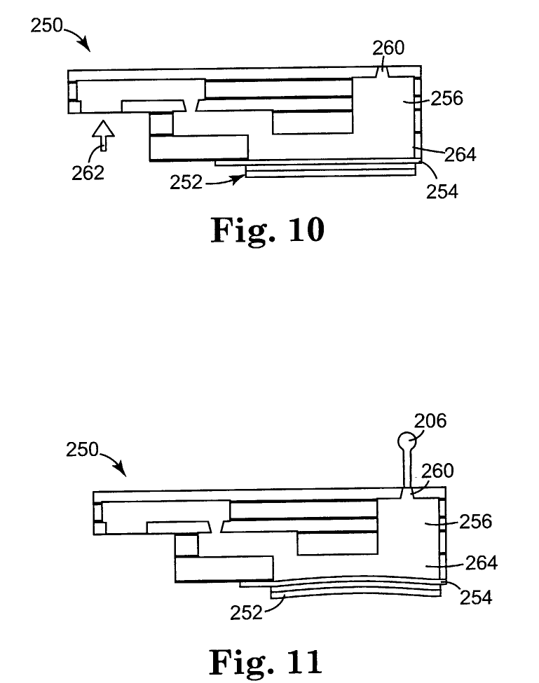

Nozzle 210 of FIGS. 4-9 is a top-ejecting nozzle, in that orifice 228 is located above resistor 216. Other nozzle configurations, such as side-ejecting configurations, are also known. Additionally, FIGS. 10-11 show an example of a piezoelectric nozzle 250. Nozzle 250 uses piezoelectric transducer 252, shown in an undeflected configuration in FIG. 10, to push and pull diaphragm 254 adjacent firing chamber 256. Upon application of electricity, the resulting physical displacement (FIG. 11) of transducer 252 and diaphragm 254 ejects droplet 206 through orifice 260. Refill fluid 262 is drawn through channel 264 for subsequent drop formation and ejection. Nozzle 250 thus mechanically moves the mass of diaphragm 254 and the fluid in firing chamber 256. Mechanical manufacturing processes are used to create nozzle 250, generally resulting in relatively lower nozzle or orifice density compared to thermal nozzles such as nozzle 210, but both thermal and piezoelectric nozzles 135 are contemplated according to embodiments of the invention.

Control circuit 105 precisely controls the amount of medication or other fluid administered to the eye from nozzles 135. The size of each droplet 206 depends on the known size of the ejection structure described above with respect to FIGS. 4-11, and is optionally programmed in or otherwise known to control circuit 105. Fluid ejection head 185 includes a known number of nozzles 135, and ejects or is controlled to eject at a known frequency. By multiplying together the number of nozzles, the volume ejected by each nozzle, the frequency with which droplets are dispensed, and the length of time the nozzles are activated, the amount of medication administered to the eye is precisely known. As one example, each nozzle 135 of a 50-nozzle array in ejection head 185 dispenses 130-nanogram droplets at a frequency of 6000 Hz. If head 185 is fired for one second, the total medication administered in one firing is (50 nozzles)(130 nanograms/nozzle)(6000 Hz)(1 second)(1×10⁻⁹ grams/nanogram) 0.039 grams. By controlling the frequency with which nozzles 135 are fired, the volume ejected per nozzle, the number of nozzles fired (e.g. for a 50-nozzle array, only one-half or some other number of nozzles are activated if desired), and the length of firing time, the amount of medication administered is precisely controlled according to embodiments of the invention. As will be described, each of these variables and other variables are optionally controlled through control circuit 105 based on instructions or information received from a remote location, such as a health-care provider or other medical professional.

Embodiments of the invention thus provide eyedropper 100 for directing fluid toward an eye of a patient. Eyedropper 100 comprises reservoir 125 for holding fluid to be directed toward the eye of the patient, nozzle 135 fluidly coupled with reservoir 125, heating element 216 adapted to form a bubble in the fluid and to eject drop 206 of the fluid through nozzle 135 toward the eye of the patient, and eye cup 200 for maintaining a generally constant distance between nozzle 135 and the eye of the patient. Second portion 155 of device 100, which is generally in the form of a cartridge, such as an ink jet pen, is operably coupled with eye cup 200 and includes at least heating element 216 and nozzle 135 as a part thereof.

According to additional embodiments of the invention, device 100 is a microprocessor-controlled device for directing a medical substance toward an eye. The device includes a thermal pump, as referenced above, for directing the medical substance toward the eye. Eye cup 200 maintains a minimum distance between the thermal pump and the eye of the patient. A microprocessor controls the thermal pump, according to embodiments of the invention. The microprocessor is, or is included in, control circuit 105, for example.

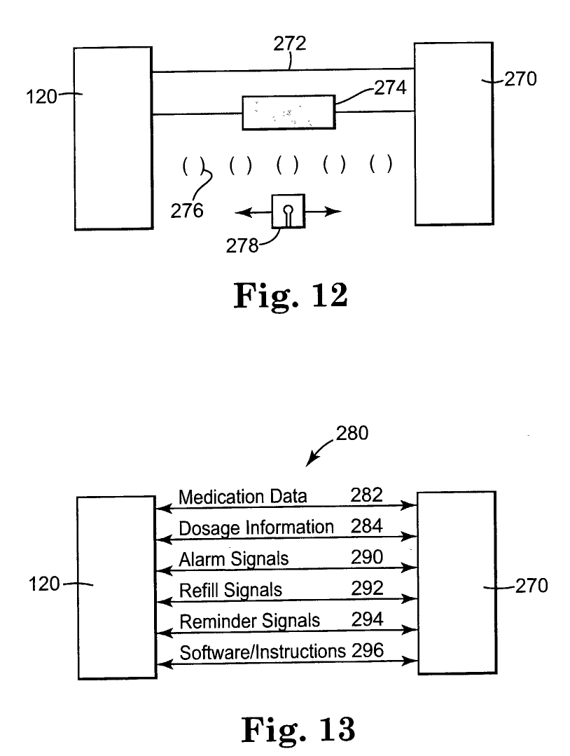

As shown in e.g. FIG. 12, communicator 120 communicates signals or other information between device 100 and location or station 270 remote from device 100. Station 270 comprises a processing device, computing device or other device capable of receiving the signals or information, and optionally is located at a remote medical provider or other location. Physicians, nurses, pharmacists, other medical professionals, or other persons at or associated with station 270, are among those with whom communication via communicator 120 is contemplated according to embodiments of the invention. Hardwired communication 272, for example using a docking station, communication 274 over the Internet or other network, and wireless communication 276, e.g. cellular, radio, infrared and other wireless forms, are examples of specific forms of communication according to embodiments of the invention. Communication using portable data storage medium 278, such as a floppy diskette, CD, optical disk or other disk, or other storage medium, is also contemplated.

A wide variety of information is capable of being communicated between communicator 120 and remote location 270, as schematically represented at 280 in FIG. 13. Data, electric signals, and instructions, for example, are among the types of information that are communicated according to embodiments of the invention. According to one example, control circuit 105 communicates such information in the form of medication data 282 to station 270, for example data about the dosage, timing, frequency and/or type of medication dispensed by device 100. Control circuit 105 optionally includes a counter, to track and communicate the number of times medication has been administered. In response to the medication data, station 270 optionally transmits, and control circuit 105 optionally receives, new dosage signals or information 284 from station 270. Dosage signals or information 284 are considered to be another form of medication data 282. Thus, according to one example, if supply of a first eye medication or other medication is depleted, or if a course of treatment using such medication is completed, new dosage information 284 transmitted from station 270 includes a direction for device 100 to switch automatically to a second eye medication. In the case of depleted medication, control circuit 105 is optionally directed to shut down device 100 or otherwise indicate to a user that an inadequate quantity of medication remains or is being dispensed. If a certain treatment protocol requires alternating use of two or more eye medications or other medications, or mixing of different medications, new dosage information 284 optionally directs control circuit 105 to alternate or mix the medications automatically and provide an indication or reminder to the user at the appropriate time. Reservoir 125 is divided-into two or more separate chambers 126, 128, as referenced previously, for this purpose or other purposes. Control circuit 105 itself also is optionally programmed to automatically change the dosage or type of medication dispensed by device 100, without direct interaction with station 270, according to embodiments of the invention. Medication data 282 and dosage information 284 optionally are communicated from communicator 120 to station 270, from station 270 to communicator 120, or in both directions.

Information 280 and/or medication data 282 also optionally include alarm signals 290. If ejection of medication by device 100 is unacceptable for some reason, for example if the user has missed one or more doses, administered too much medication, or otherwise has not followed a prescribed course of treatment, or if device 100 is malfunctioning or is out of medication, device 100 communicates electric signals regarding the unacceptable condition to station 270. According to one embodiment, such signals constitute alarm signals 290. According to other embodiments, station 270 receives electric signals regarding use of device 100, itself determines that an unacceptable condition exists, and then generates alarm signals 290 for communication back to device 100. According to one embodiment, station 270 compares use of device 100 with a prescribed treatment protocol, and generates alarm signals or an alarm in the event of a discrepancy. An alarm is created, at either station 270, device 100, or both. According to other embodiments, control circuit 105 itself generates an alarm based on unacceptable ejection of medication or other unacceptable condition, independently of station 270. Verification, accountability and reliability are provided.

According to other examples, medication refill signals 292 are a type of medication data 282 communicated between device 100 and station 270, in either or both directions. For example, control circuit 105 determines that one or more types of medication within reservoir 125 are almost depleted, for example by counting the number of uses of device 100 or by more directly sensing the fluid level within reservoir 125, for example with optional sensors. Control circuit 105 then communicates that information in the form of refill signals 292 to station 270, via communicator 120. Station 270 then initiates an appropriate response, e.g. directing that a refill prescription be issued or filled, notifying the user of device 100 and/or shutting device 100 down. Alternatively, or additionally, uninterpreted data is communicated from communicator 120 to station 270, and station 270 communicates refill signals 292 to communicator 120.

Station 270 optionally communicates reminder signals 294 as a form of medication data 282, for example to remind a user of device 100 to administer medication. Device 100 optionally includes visual, audio, and/or tactile indicators to remind the user that a dose is required or otherwise provide a desired indication to the user. Additionally, device 100 optionally communicates reminder signals 294 to station 270, for example to remind a pharmacist or other medical professional that interaction with the user of device 100 should be initiated.

Software or instructions 296 for programming or operating control circuit 105 optionally are communicated to or from remote station 270, for example upon initial use of device 100, upon a change in prescription or treatment protocol, upon software upgrade, or at another desired time. Control circuit 105 controls device 100 in accordance with the new software or instruction, according to one embodiment, with appropriate notification to the user if needed. According to embodiments of the invention, dosage amounts are optionally ramped up or down automatically over a number of days or other time period, medications are optionally changed automatically, and reminders optionally provided automatically. These and/or other features optionally cause device 100 to be perceived as a “smart” eyedropper device and provide a number of features and advantages.

Thus, device 100 is part of an eye medication communication and application system according to an embodiment of the invention. The system includes an applicator, which is or is part of device 100, for introducing eye medication to an eye. The system also includes a computing device, for example control circuit 105 or a part thereof, for controlling the applicator. Communicator 120 is operably coupled with the computing device. Station 270, remote from the applicator and the computing device, is adapted to communicate medication data to the computing device via communicator 120 or receive medication data from the computing device via communicator 120.

In use, according to one embodiment, control circuit 105 is programmed or otherwise adapted to control dispensing of one or more types of eye medication or other substance, for a predetermined period of time and in a predetermined dosage. The user places device 100 near the eye. Eye cup 200 guides the user to keep a desired distance between fluid ejection head 185 and the eye, e.g. generally prevents the user from moving ejection head 185 too close to the eye. The user then depresses dosage switch 115 to eject the predetermined type and dosage of medication toward the eye. Alternatively, medication is ejected for as long as switch 115 is depressed, so that the user can directly control dosage. Control circuit 105 optionally records, e.g. in an associated memory, data regarding the ejection, for example the amount and type of medication dispensed, as well as the time of day and/or date of dispensing. Communicator 120 optionally communicates data regarding the ejection to remote location or station 270. Alarm indications, refill conditions, verifications, new dosage instructions, new operating instructions or software, and other information and data are optionally communicated between station 270 and device 100. Embodiments of device 100 thereby precisely control the type and amount of medication dispensed, automatically verify that a proper protocol is being followed, automatically change the type of medication dispensed, are easier or more desirable for child users and/or provide better verification that a child or other user is administering medication properly, and provide other optional advantages as may desired for a particular patient or situation.

Embodiments of the invention also provide a method of eye medication ejection control. The method includes ejecting drops of eye medication using an ejector, for example a thermal ejector, in eyedropper 100, transmitting data regarding the ejected eye medication to location 270 remote from eyedropper 100 and/or receiving data at eyedropper 100 from location 270 or other location remote from eyedropper 100, and controlling ejection of the eye medication, using e.g. control circuit 105, based on the transmitted and/or received data. The method also optionally includes adjusting the amount of eye medication dispensed, adjusting the type of eye medication dispensed, alternating between at least two different types of eye medication, indicating to a user that eye medication should be dispensed, and/or electronically verifying that dispensing is occurring according to a predetermined protocol.

Embodiments of the invention also include one or more computer-readable media having stored thereon a computer program that, when executed by a processor, causes eye medication ejection control, alarm generation, medical information communication, and/or the other features and capabilities described herein.

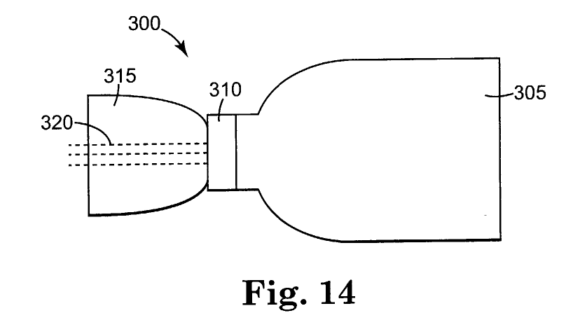

Device 100 is of any desired shape, according to embodiments of the invention. According to one specific embodiment, illustrated in FIG. 14, device 300 is generally in the more commonly perceived form of an eye drop bottle. Device 300 includes generally bottle-shaped member 305, which houses or supports a fluid reservoir, fluid ejection head and/or other features illustrated and described with respect to e.g. portion 155 of FIGS. 2-3. Member 305 supports frame 310, which is akin to portion 150 and supports features such as a dosage dispensing switch. Eyecup 315 maintains a generally fixed distance between the ejection head and the user's eye, for application of drops 320.

Although the present invention has been described with reference to certain specific embodiments, those skilled in the art will recognize that changes may be made in the form and detail of those specific without departing from the spirit and scope of the invention. For example, the drawings associated with this disclosure are not necessarily to scale. Embodiments of the invention are capable of use with both human and veterinary patients. Medications and substances other than eye medications and substances are also contemplated for use. Communicator 120 can be eliminated and control provided solely by dosage switch 115 and/or other associated switches, dials or other controls, in accordance with manual input by a user. Other aspects of the invention will be apparent to those of ordinary skill upon reading this disclosure.

What is claimed is:

1. An eyedropper for directing fluid toward an eye of a patient, the eyedropper comprising:
a reservoir for holding fluid to be directed toward the eye of the patient;
a nozzle fluidly coupled with the reservoir;
an element adapted to eject a drop of the fluid through the nozzle toward the eye of the patient; and
an eye cup for maintaining a generally constant distance between the nozzle and the eye of the patient.

2. The eyedropper of claim 1, wherein the element and the nozzle are disposed in a cartridge operably coupled with the eye cup.

3. The eyedropper of claim 1, wherein the element comprises a thin-film resistor adapted to form a bubble in the fluid.

4. The eyedropper of claim 1, wherein the nozzle is one of an array of nozzles.

5. A microprocessor-controlled device for directing a medical substance toward an eye of a patient, the device comprising:
a thermal pump for directing the medical substance toward the eye of the patient;
structure for maintaining a minimum distance between the thermal pump and the eye of the patient;
a microprocessor for controlling the thermal pump; and
a communicator, operably coupled with the microprocessor, for communicating signals between the device and a location remote from the device.

6. The device of claim 5, wherein the communicator comprises a communications port.

7. The device of claim 5, wherein the communicator comprises a USB port.

8. The device of claim 5, wherein the communicator comprises a wireless communication device.

9. The device of claim 5, wherein the thermal pump and the microprocessor are located in a common housing.

10. The device of claim 5, wherein the microprocessor is adapted to control the dosage of the medical substance directed toward the eye.

11. The device of claim 5, wherein the microprocessor is adapted to control the thermal pump to direct thousands of drops of the medical substance per second toward the eye.

12. The device of claim 5, wherein the signals are alarm signals.

13. The device of claim 5, wherein the signals are dosage signals or medication-refill signals.

14. An eye medication communication and applicator system, comprising:
an applicator for introducing eye medication to an eye;
a computing device for controlling the applicator;
a communicator operably coupled with the computing device; and
a station remote from the applicator and the computing device, the station being adapted to communicate medication data to the computing device via the communicator or receive medication data from the computing device via the communicator.

15. The system of claim 14, wherein the applicator comprises a resistor for ejecting drops of the medication toward the eye.

16. The system of claim 14, wherein the applicator comprises a piezoelectric device for ejecting drops of the medication toward the eye.

17. The system of claim 14, wherein the computing device and the station are adapted to communicate via the Internet.

18. The system of claim 14, wherein the computing device and the station are adapted to communicate via a wireless communication system.

19. The system of claim 14, wherein the computing device is adapted to receive new dosage information from the station and to change the dosage of eye medication introduced by the applicator.

20. The system of claim 19, wherein the eye medication is a first eye medication, further wherein the computing device is adapted to automatically control the applicator to dispense a second eye medication instead of the first eye medication in response to the new dosage information.

21. A method of medication alarm generation, the method comprising:
ejecting drops of medication using an ejector in a handheld housing;
generating electric signals regarding the ejection;
communicating the electric signals from the handheld housing toward a remote location; and
generating an alarm if the electric signals represent unacceptable ejection of the medication.

22. The method of claim 21, wherein the ejecting step comprises using a thermal ejector comprising a heating element.

23. The method of claim 21, wherein the generating of the alarm occurs at the remote location.

24. The method of claim 21, further comprising receiving at least one alarm signal from the remote location, the alarm signal triggering the alarm at the handheld housing.

25. The method of claim 21, wherein the unacceptable ejection is due to the amount of medication ejected.

26. The method of claim 21, wherein the unacceptable ejection is due to the timing of the ejection of the medication.

27. The method of claim 21, where the communicating occurs via a communications port disposed at the handheld housing.

28. The method of claim 21, where the communicating occurs via a removable data storage medium.

29. The method of claim 21, wherein the ejecting comprises ejecting medication toward an eye of a patient.

30. A method of eye medication ejection control, the method comprising:
ejecting drops of eye medication using an ejector in an eyedropper;
transmitting data regarding the ejected eye medication to a location remote from the eyedropper and/or receiving data at the eyedropper from a location remote from the eyedropper; and
controlling ejection of the eye medication based on the transmitted and/or received data.

31. The method of claim 30, wherein the ejecting step comprises using a thermal ejector comprising a heating element.

32. The method of claim 30, wherein the controlling step includes adjusting the amount of eye medication dispensed.

33. The method of claim 30, wherein the controlling step includes adjusting the type of eye medication dispensed.

34. The method of claim 33, wherein the controlling step includes alternating between at least two different types of eye medication.

35. The method of claim 30, wherein the controlling step includes indicating to a user of the eyedropper that eye medication should be ejected.

36. The method of claim 30, further comprising electronically verifying that ejection is occurring according to a predetermined protocol.

37. One or more computer-readable media having stored thereon a computer program that, when executed by a processor, causes eye medication ejection control according to the following method:
ejecting drops of eye medication using an ejector in an eyedropper;
transmitting data regarding the ejected eye medication to a location remote from the eyedropper and/or receiving data at the eyedropper from a location remote from the eyedropper; and
controlling ejection of the eye medication based on the transmitted and/or received data.

38. One or more computer-readable media having stored thereon a computer program that, when executed by a processor, causes medication alarm generation according to the following method:
ejecting drops of medication using an ejector in a handheld housing;
generating electric signals regarding the ejection;
communicating the electric signals from the handheld housing toward a remote location; and
generating an alarm if the electric signals represent unacceptable ejection of the medication.

39. An eyedropper, comprising:
means for ejecting drops of eye medication using a thermal ejector;
means for transmitting data regarding the ejected eye medication to a location remote from the eyedropper and/or receiving data at the eyedropper from a location remote from the eyedropper; and
means for controlling ejection of the eye medication based on the transmitted and/or received data.
